(19) United States
(12) Patent Application Publication
(10) Pub. No.: US 2023/0172639 A1
(43) Pub. Date: Jun. 8, 2023

(54) ENCLOSURE DEVICE FOR BONE REPLACEMENT AND METHOD OF MANUFACTURING THE SAME

(71) Applicant: OSSFORM PTY LTD, Cairns, Queensland (AU)

(72) Inventor: Neil Meredith, Cairns (AU)

(21) Appl. No.: 17/997,390

(22) PCT Filed: Apr. 30, 2021
(86) PCT No.: PCT/AU2021/050392

§ 371 Date: Oct. 28, 2022

(30) Foreign Application Priority Data
May 1, 2020 (GB) 2006465.5

(51) Int. Cl. A61B 17/56; A61F 2/28

(52) U.S. Cl. CPC A61B 17/56; A61F 2/2846; A61B 2017/00526

(57) ABSTRACT

A hollow bone enclosure (16), of resorbable polymer, such as poly lactic or poly glycolic acid. The hollow bone enclosure comprises an inner contact portion (18), for insertion into a bone defect (not shown) and arranged to make an intimate fit with the surface of the defect, and an outer closure portion.

The present invention relates to a device for promoting the replacement of bone.

Bone may be lost for a number of reasons, including disease, trauma or neoplasm. There may also be a congenital absence of bone in some patients.

Replacement of missing bone is often desirable to restore function or strength, and may be required for aesthetic reasons, particularly in dental or maxillofacial cases.

Previously considered techniques for replacement of missing bone typically involve surgery to graft a piece of bone to the deficient site. An artificial framework may also be used, such as an open membrane, plate, trough or channel, to support introduced bone or artificial bone replacement material or bone growth-inducing material.

However, prior approaches have a number of disadvantages. Firstly, a high level of surgical skill is necessary. Secondly, accurate placement of the augmentation or replacement material, which typically comprises crushed or particulate bone or bone substitute, is very difficult to achieve, and it can move after placement. Because of this the support framework typically requires fixing in place. However, the framework may need to be removed after healing.

U.S. Pat. publication number US 2010/0291508 describes a trough device used for a bone augmentation procedure. The device comprises a rib portion and two sidewalls extending from the rib. In use the edges of the sidewalls are arranged to rest on a portion of a human jaw bone to be treated.

In some previously considered techniques, solid grafts are sometimes used to repair a defect. These may comprise the patient’s own bone and may be taken from another part of the body and cut to fit. Alternatively, a block of synthetic material of approximately the correct size may be cut, ground or milled. The cutting or grinding process may be manual or automated, for example using a CAD-CAM technique. One of the problems of solid or dense blocks of material is the inability of new blood vessels, repairing and regenerating cells and new bone to penetrate the solid material.

Implantable medical devices are commonly used to replace missing or damaged body parts, tissue and bones. Typically, these devices are prefabricated and are available in a range of sizes and shapes designed to achieve a best fit with the damaged or missing tissue. Such devices may be free standing or anchored in place with screws, pins, bolts or cements.

It would be most advantageous to be able to design a custom implant specific to each patient and situation, providing an optimal interface between the prosthesis and the patient’s tissue as well as restoring the original or ideal geometry for aesthetics, and function. Such an implant may serve a therapeutic function not only replacing missing tissue but having a structure that encourages healing and formation of natural tissues.

Custom made prosthetic devices may be made by taking a physical impression of the body interface as a negative and using such an impression as a mould to create a positive model onto which the prosthesis can be manufactured before fitting to the patient, e.g. replacement tooth dentures. To do this requires direct access to the surface onto which the prosthesis may be made. The fitting surface of the implant is created from the impression and the external tissue surface replacing the missing tissues is designed by a person skilled in the art or more recently Computer Assisted Design programs.

Such prosthetic devices may be made from casting, sintering, moulding, milling or rapid prototype methods.

It is now possible using radiological techniques and visual scanning methods to produce three dimensional images of hard (bony) and soft tissues of parts of the body. This can enable three-dimensional models of the body parts to be constructed from such digitized information either as surface rendered or volume-based data by a range of rapid prototyping techniques. European patent EP 0 756 735 B discusses such a process; the production and the modification of such replica models to assist the surgeon in surgical techniques by fabrication of a template that may be used to guide surgery.

Embodiments of the present invention aim to address at least some of the shortcomings of the prior art.

The present invention is defined in the attached independent claims, to which reference should now be made. Further, preferred features may be found in the sub-claims appended thereto.

According to one aspect of the present invention, there is provided an enclosure device for the treatment of a bone, the device comprising a contact portion for contacting a selected area of bone in use, and at least one closure portion, the contact portion and the closure portion together defining a substantially enclosed volume, wherein a surface profile of the contact portion matches a surface of the selected area of bone, such that when the enclosure device is placed on the bone the contact portion forms a close or intimate engagement with the surface of the selected area of bone.

In a preferred arrangement the enclosure device is substantially hollow.

The contact portion may comprise a contact surface that, in use, extends across an area of bone surface. Preferably substantially the entire contact portion is arranged in use to make an intimate fit with the bone surface. In order to make an intimate fit with the bone surface the contact surface of the contact portion may be a substantially exact match to the surface of the bone.

The contact portion may be arranged to extend across an area of bone surface comprising a recessed portion of bone.

In a preferred arrangement the contact portion and/or the closure portion is at least partly perforated or porous to provide a plurality of apertures or pores for allowing the ingress of blood, cells and forming bone.

The contact portion and closure portion may be integral. Alternatively, the closure portion may be separable from the contact portion and may comprise a lid.

The contact portion and the closure portion may be formed as separate elements and may be attached together before, during or after installation of the enclosure device.

The contact portion and the closure portion may be attached together by attachment means or by a friction fit. The attachment means may comprise one or more of clips, pins, screws, lugs, tabs or latches.

The enclosure device may be provided with a flange portion for mounting on the bone surface. The flange portion may be arranged in use to extend from the contact portion or from the closure portion and may be shaped so as to rest on the bone surface. The flange portion may be provided with one or more mounting features to assist in mounting the enclosure device on the bone.

The enclosure device may be provided with one or more support portions for supporting the device in use. The support portions may comprise tabs or wings, which may have mounting features for mounting on the bone.

The mounting features may comprise holes or brackets for receiving fasteners, for example such as screws.

The enclosure device may have one or more internal stiffening members for maintaining rigidity of the device.

The enclosure device may be provided with one or more loading apertures to allow material to be loaded into the device.

The enclosure device may comprise a biocompatible material such as titanium and/or one or more of its alloys, one or more ceramics, such as calcium hydroxy apatite, or one or more polymers such as poly lactic or poly glycolic acids, or combinations thereof.

According to another aspect of the invention, there is provided a method of manufacturing an enclosure device for the treatment of a bone, the method comprising creating electronically a design for the enclosure by generating a virtual model of a selected area of bone, defining a margin for the bone enclosure device, using the defined margin to determine at least one surface profile of the enclosure device from the virtual model.

The method may include using the electronically created design to guide the operation of an automated 3-D printer or rapid prototyping apparatus.

The enclosure device may comprise a contact portion for contacting the selected area of bone in use and at least one closure portion. Preferably the method comprises using the defined margin to determine a surface profile of the contact portion and/or a surface portion of the closure portion.

The step of defining a margin for the bone enclosure device may comprise manually drawing an outline of the margin on an electronic touch sensitive interface. Alternatively, or additionally software may be used to define a contact surface, e.g. by copying the virtual bone model surface.

The invention also includes a computer program product comprising computer program code arranged in use to perform a method according to any statement herein when said computer program code is run on a computer.

The computer program code may be embodied in a computer readable medium.

The invention may comprise any combination of the features or limitations referred to herein, except any such combination in which the features are mutually exclusive.

Embodiments of the present invention will now be described by way of example only, with reference to the accompanying drawings, in which:

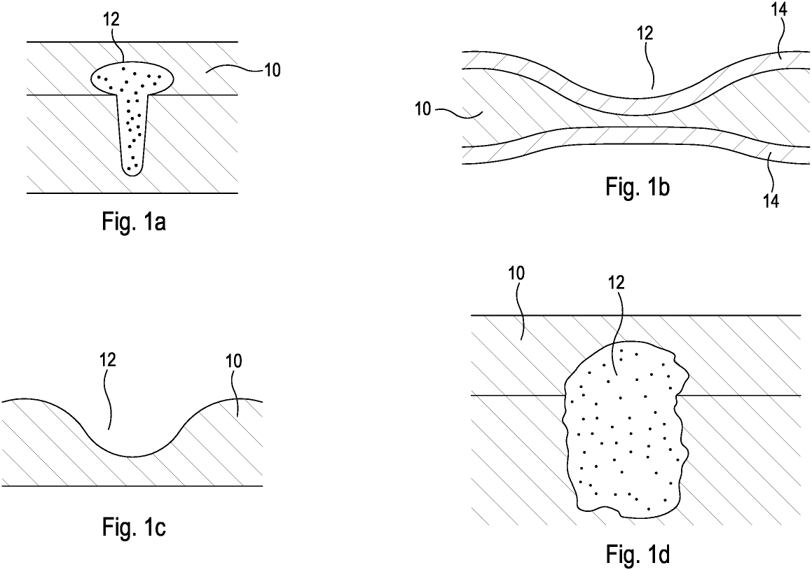

FIGS. 1(a) to 1(d) show schematically several common bone defects in humans;

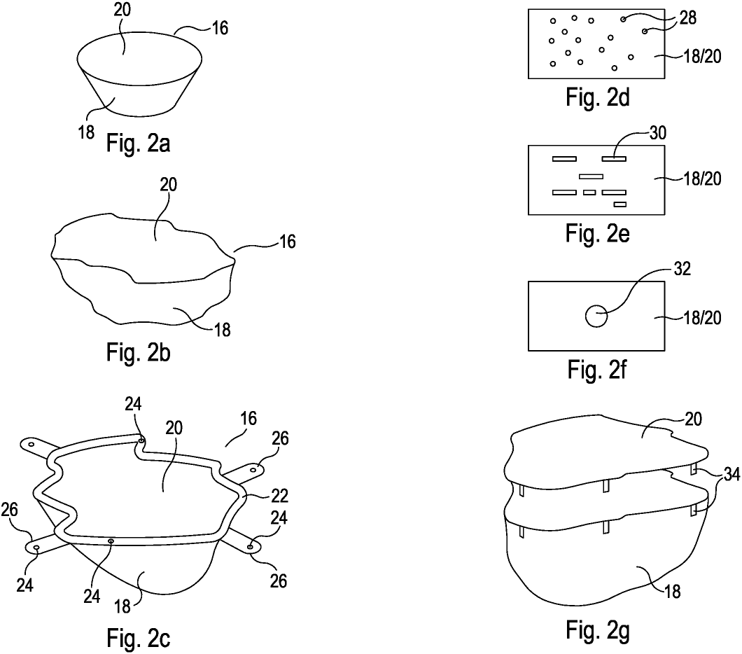

FIGS. 2(a) to 2(g) show schematically examples of enclosures in accordance with embodiments of the invention; and

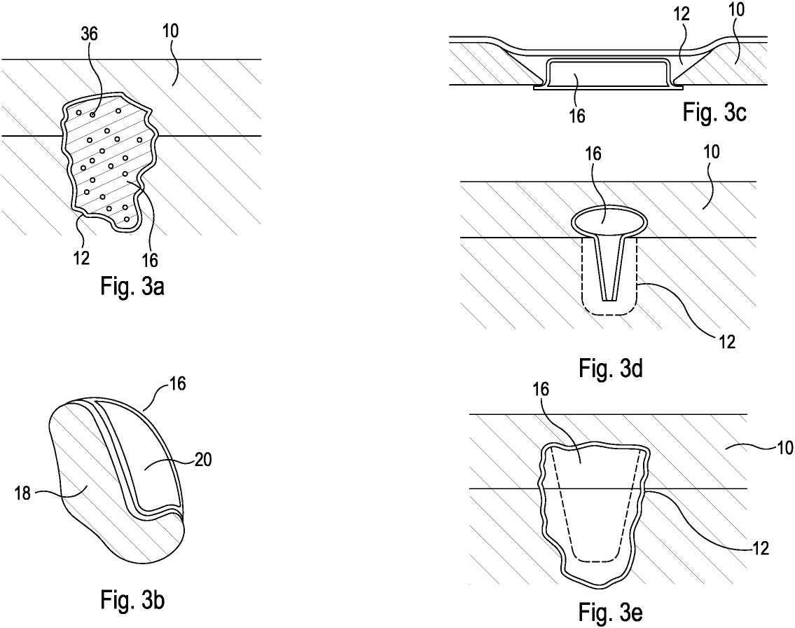

FIGS. 3(a) to 3(e) show schematically examples of bone defects restored or rectified with bone enclosures in accordance with embodiments of the present invention.

Embodiments of the present invention described below comprise a device suitable for use in surgery on humans or animals to assist or replace bone augmentation and grafting procedures. In its basic form the device comprises a hollow enclosure suitable to be fitted onto a defect site in the bone. In most cases the enclosure will comprise an irregularly shaped, custom-made device that is designed to fit exactly the topography of the bone defect. There may be optional additional features as will be described below.

In most cases the enclosure is substantially hollow and has an inner, contact portion, which is arranged to contact the defective bone surface, and an outer, closure portion which is intended to become covered in skin, mucosa or soft

tissue. The contact portion will typically be irregular and have a positive profile surface designed to match the negative surface of the defective bone site, in order to make an intimate fit therewith. The surface of the bone with which the contact portion fits will typically comprise a recess in a portion of bone. The outer, or closure, portion will typically be smooth in order to restore the contour of the defect to that of the original and adjacent bone.

FIGS. 1(a) to 1(d) show schematically some common bone defects. In each case the bone is identified as 10, and a defect is denoted by the numeral 12.

FIG. 1(a) shows a tooth extraction socket, FIG. 1(b) shows an area of the human palate where bone has been lost, with soft tissue being denoted by numeral 14, FIG. 1(c) shows a defect in a bone and FIG. 1(d) shows a more irregular defect in which bone has been lost due to trauma or disease.

FIGS. 2(a) to 2(g) show various embodiments of bone enclosure, in accordance with the present invention.

In FIG. 2(a) a hollow bone enclosure, of resorbable polymer, such as poly lactic or poly glycolic acid, is shown at 16 comprising an inner contact portion 18, for insertion into a bone defect (not shown) and arranged to make an intimate fit with the surface of the defect, and an outer closure portion 20.

FIG. 2(b) shows an embodiment in which the contact portion 18 has an irregular shape which has been contrived to be an exact fit with the surface of a bone defect (not shown).

A major benefit of bone enclosure devices according to the present invention is that the surface of the contact portion 18 is made to exactly mirror the surface of the bone defect, thereby acting as a plug. Because of this, externally applied forces in any direction are resisted and movement of the enclosure is inhibited. This inherent stability can be further increased for the enclosure by extending a flange from the contact portion or closure portion onto the bone beyond the margin of the defect.

FIG. 2(C) shows an embodiment of the invention in which the enclosure 16 is provided with a flange 22 extending a few mm (typically 1-10 mm) around the top edge of the enclosure 16.

The flange 22 enhances stability and prevents rotation against locally applied loads. The thickness and width of the flange 22 may vary according to implementation. It can also be provided with screw holes 24 which can be used to further stabilise the enclosure by placing screws (not shown) through the flange 22 into the adjacent bone.

The enclosure may include a number of additional, optional features. For example, the enclosure may be provided with one or more brackets or wings 26 extending from the closure portion 20 or contact portion 18, or from the flange 22. The wings 26 can extend over the bone (not shown) and may incorporate screw holes 24.

The internal part of the enclosure is hollow. This is to enable it to fill naturally with new bone. Alternatively, it can be filled with an artificial material that can be used as a scaffold for new bone formation or a factor to accelerate bone growth. It may be desirable to increase the stiffness of the contact and closure portions of the enclosure. This can be achieved by having stiffeners or supports such as struts (not shown) connected to and extending between the contact and closure portions. This is of particular benefit if there is no material inside the enclosure or if the wall or either portion is relatively thin.

The hollow enclosure of the present invention enables a large part of the volume of the defect to be, or to become, filled with new bone in a stable environment.

In order to enable blood, blood vessels and cells to penetrate the enclosure the surface of the contact portion is perforated with a series of regularly or irregularly shaped, uniformly spaced or randomly placed holes. This will allow blood to fill the enclosure and completely saturate any material placed inside the enclosure.

FIGS. 2(d) to 2(f) show a part of the contact portion 18, respectively with a plurality of randomly distributed perforations 28, regular holes 30 and a single large hole 32.

A regenerative material may be placed inside the enclosure at the time of surgery. Such material may be crushed or particulate bone or artificial materials such as hydroxy apatite designed to create a scaffold for new bone formation. This is not essential as blood alone may fill the space and has the capacity to form new bone. On occasions it may be useful to have a hole or port in the contact or closure portion, or else in both, which may be used to load the enclosure with bone augmentation materials or similar.

The thickness of the walls of the contact and closure portions may vary within a single enclosure and/or between different enclosures. The three principal factors which will influence the thickness of the walls of each portion 18 and 20 are strength, material and function. The primary function of the enclosure is to provide a stable space to enable new bone to form undisturbed during healing and support the forming tissue and material in an enclosed space to aid surgical handling and placement. It is beneficial for the walls of the enclosure to be as thin as possible whilst maintaining strength. The enclosure may be designed to be load-bearing, though it need not do so in each case.

New bone takes time to form and this varies depending on a number of factors including the site, the patient’s age, medical history, local factors such as materials and growth factors designed to accelerate bone formation. The time taken may vary approximately from four weeks up to a year. The longer the bone is left to heal the higher its mineral content and the greater its ability to distribute applied loads, such as are encountered when walking or chewing for example, without damage. The enclosure should therefore remain in place until the bone has become mature and able to support loads. The enclosure is not designed to be removed because the new bone will form around it. It must therefore be made from a biocompatible material. Commonly used materials include metals, such as titanium and its alloys, ceramics such as calcium hydroxyapatite and polymers such as poly lactic and poly glycolic acids. The bone enclosure may be fabricated from any one or combination of these. An advantage of ceramics and polymers is that they may dissolve and resorb after a time to become incorporated into the newly formed bone. If the material is made from a ceramic or polymer a thinner wall may be desirable as it will resorb more quickly.

The closure portion of the bone enclosure will be in contact with epithelium, soft tissue, mucosa or connective tissue. The soft tissue should not grow into the newly forming bone but if possible allow the blood supply and nutrients to spread into the healing hard tissue.

The contact portion 18 and closure portion 20 of the enclosure 16 may comprise separate elements or may be made as one and may comprise the same or different materials.

FIG. 2(g) shows an outer lid part 20a comprising the closure portion and an internal body part 18a comprising the contact portion.

If the closure and contact portions are made as separate parts they may fit together by a push/friction fit created by one part being smaller than the other. Alternatively clips, pins, lugs, tabs, screws or the like (not shown) may be used to attach the portions. In FIG. 2(g) each of the portions 18a and 20a is provided with lugs 34 for a mutual friction fit between the portions.

The enclosure may be manufactured using any one or a combination of well proven technologies. The enclosure may be prefabricated in a range of standardised shapes and may be adjusted by bending, adapting or grinding at the time of surgery.

Such production technologies include for metals; casting, punching, pressing and sintering; for polymers moulding, pressing, extruding, spraying or printing; and for ceramics pressing or sintering.

The enclosure may also be tailor- or custom-made from a pattern of the defect. Such a pattern may be a simple handmade template or may be computer generated from a digital image of the defect. The image may be produced by contact, laser or optical scanning or by three-dimensional radiographic scanning. Computer aided design software can be used to generate a model of the enclosure. The model can be optimised by freehand or automated design to include material, mechanical properties, stress distribution, effect of loading, and/or geometric features.

The computer generated model of the custom enclosure can then be manufactured using computer assisted manufacturing methods such as milling, moulding, printing, sintering and/or stereo lithography. Such techniques are readily available for biocompatible materials including ceramics, polymers and metals.

Advantages offered by computer assisted manufacturing include a high degree of control over material shape and thickness, the ability to develop complex geometries and features and the ability to build-in analytic and diagnostic features.

In previously considered techniques troughs, trays and shells have been used to support bone grafts. All suffer from the disadvantage that there is an absence of support for the graft material, which may be a gel or small or large particles, during placement. This can cause the material to fall out. Enclosures in accordance with the above described embodiments overcome this problem.

Another of the problems with using a trough, tray or shell to support a bone graft is stability and accuracy during placement. Whether the trough, tray or shell is prefabricated, formed by hand, or by a tool or machine (CAD-CAM), it is often difficult to locate accurately on the defective bone face. In addition, the shell must be very rigid to allow new bone to form without experiencing external pressure which would disrupt the healing process.

A trough, shell or tray will only contact the bone on its edges and it will, therefore, lack rigidity and stiffness. In contrast to this a bone enclosure according to embodiments of the present invention contacts the bone defect on substantially its entire internal face. Furthermore, the box-like structure is considerably stiffer than open-sided structures. A key benefit of this is the prevention of load forces being applied to the healing tissue and newly forming bone. A chewing load from the teeth is an example of such a force.

FIGS. 3(a) to 3(e) show examples of bone defects that have been restored using bone enclosure devices in accordance with embodiments of the present invention.

In FIG. 3(a) a typical irregular bone defect 12 in a part of a bone 10 has been restored by inclusion of a bone enclosure 16. The enclosure 16 has in this case been filled with bone material 36. FIG. 3(b) shows the enclosure 16 of FIG. 3(a) with contact portion 18 and closure portion 20.

FIG. 3(c) shows an enclosure 16 located in a bone 10 in the palate region of an upper human jaw.

FIG. 3(d) shows an enclosure 16 that has been used to replace the root of a human tooth.

FIG. 3(e) shows an enclosure 16 that has been used to restore an irregular bone defect 12.

An important benefit of the bone enclosure device described is that the contact portion 18 is able to make an intimate fit with an internal surface of a bony defect. The bony defect may comprise an opening that is smaller than the body, or cavity, of the defect itself. Because of this, the contact portion 18 of the enclosure device should ideally be free from undercuts or it may not be able to adapt completely to the surface of the bony defect

In accordance with a preferred embodiment of the invention, there follows a description of a method of manufacture of a bone enclosure device.

The method involves taking digital image information of an object, converting it into a suitable format, using such information in combination with computer assisted information and manual and automated design to construct a design for an implantable medical device suitable for manufacture by a rapid prototyping apparatus.

To construct a custom made implantable medical device at least the following elements need to play an interactive role:
Digital, scanned or radiographic data of the body part must be constructed in an appropriate format to enable construction and design modification of the implantable device;
Software containing data on mechanical design principles, physical properties of materials for both the body and the implantable device to set the shapes, tolerances, limits and geometry and designs of the implantable device in relation to its function, location and application;
External input from a person skilled in the art (i.e. a surgeon) is used to design and refine the implant shape and geometry guided by the software mentioned above. This may be performed by manual input to the software using a digitizing system such as a mouse, pen, tablet or keyboard;
The implant design created by the combination of the software and the external input, on the basis of the data of the body part is converted from a three dimensional image file into a format suitable for manufacturing by an appropriate rapid prototyping method.

European patent EP 0 756 735 B describes the production of a virtual 3-D model of a body part from digital imaging*- information using rapid prototyping. This model may be positive (ie a direct copy) or a negative (ie a mould) which would fit the original accurately. Variations to the model are described which include the inclusion of holes or positive features which may be used in the fabrication of a physical template to assist a surgeon in drilling implants. A further variation is described in which the model is overextended to provide a physical template onto which a metal foil or membrane may be adapted to support a graft on a patient’s actual bone.

The method described in this patent differs from the present invention, in which only a virtual model of the body part is generated in three dimensions within the computer system software as a voxel or similar file. This file is then opened in a piece of software that enables the model to be visually inspected by the person skilled in the art. It can be rotated in three directions and made solid or transparent to aid visualization. The virtual model can also be magnified and sectioned to aid further investigation.

The software is used to create an implantable device as an enclosure having an inner contact portion (bone facing) surface and outer closure portion (periosteum / tissue facing) surface to restore a bone defect created by e.g. tooth loss, disease, infection, trauma, congenital absence or neoplasm. It can also be used to create a three-dimensional membrane i.e. a two-dimensional plate or sheet of uniform or varying thickness formed into three dimensions.

The margin of the enclosure, ie the junction between the contact portion surface and closure portion surface, may be defined by freehand drawing on the virtual image by a skilled user. This is usually the margin of the defect itself in the selected area of the bone.

The contact portion surface of the device can have a range of structural features including, but not limited to: thickness variations, holes, porosity, ports etc.

The closure portion surface margins extend from the line drawn by the person skilled in the art. The surface contour of the closure portion is created as an extrapolation of the outer bone surface beyond the margin of the defect. This can be achieved by three-dimensional curve fitting routines which are known. The skilled operator may have the opportunity for manual intervention, for example to drag the surface; to lift it and/or depress it at certain points, thus manually altering the contour.

The closure portion surface of the device can have a range of structural features including but not limited to: variable thickness, holes, porosity, ports etc.

The contact and closure surfaces of the device will usually meet at the margin of the bone defect as drawn by the person skilled in the art. They may be formed as one piece or alternatively the contact portion and closure portion surfaces may be separate. They may join or be made to join in a number of ways, including but not limited to: by a recess or combination thereof, by clips or held together by screws or attachments of one kind or another.

The closure portion surface of the device may be extended beyond the margin onto natural bone. The degree of extension can be selected by the operator and may typically range from 1-10 mm. The thickness of the extension can also be varied and will typically range from 0.1-2 mm. The purpose of this extension is to assist in seating and prevent displacement and tipping. It will also distribute applied loads away from the defect area and onto sound bone.

The extensions may also have additional features that the operator can pick and place. These may be strips with holes to enable remote attachment of the device to aid stabilization.

The operator may alter the transparency, magnification and rotation of the native bone and the device to enable inspection.

The method may include incorporating algorithms that can calculate the stresses, strains and displacements of applied loads to the native bone and the device. This enables the operator to choose suitable properties for the device, including but not limited to: thickness and material.

Supports may be made between the contact and closure portions. These can provide reinforcement and act to stabilize the device. The location, number and thickness of these supports, which may be in the form of pillars, may be chosen manually by the operator or automatically by the software.

The software then generates one or more three dimensional models in a file format suitable for translation to a proprietary CAM program i.e. STL. The device is then manufactured by any of a range of rapid prototyping machines available, including but not limited to: milling, moulding, printing, sintering and/or stereo lithography. Such techniques are readily available for biocompatible materials including ceramics, polymers and metals.

The examples described above are of restoration of human bone defects. However, bone enclosures in accordance with embodiments of the present invention may also be suitable for use in treating bone defects in non-human bones.

Whilst endeavouring in the foregoing specification to draw attention to those features of the invention believed to be of particular importance, it should be understood that the applicant claims protection in respect of any patentable feature or combination of features referred to herein, and/or shown in the drawings, whether or not particular emphasis has been placed thereon.

1. An enclosure device for the treatment of a bone, the device comprising
a contact portion for contacting a selected area of bone in use, and
at least one closure portion, the contact portion and the closure portion together defining a substantially enclosed volume,
wherein a surface profile of the contact portion matches a surface of the selected area of bone, such that when the enclosure device is placed on the bone the contact portion forms an intimate engagement with the surface of the selected area of bone.

2. An enclosure device according to claim 1, wherein the enclosure device is substantially hollow.

3. An enclosure device according to claim 1 , wherein the contact portion comprises a contact surface which, in use, extends across an area of bone surface.

4. An enclosure device according to claim 1, wherein substantially the entire contact portion is arranged in use to make an intimate fit with the bone surface.

5. An enclosure device according to claims 1, wherein the contact portion is arranged to extend across an area of bone surface comprising a recessed portion of bone.

6. An enclosure device according to claims 1, wherein the contact portion and/or the closure portion is at least partly perforated or porous to provide a plurality of apertures or pores for allowing the ingress of blood.

7. An enclosure device according to claims 1, wherein the contact portion and closure portion are integral.

8. An enclosure device according to claim 1, wherein the closure portion comprises a lid separable from the contact portion.

9. An enclosure device according to claim 1, wherein the contact portion and the closure portion are formed as separate elements for attachment together before, during or after installation of the enclosure device.

10. An enclosure device according to claim 9, wherein the contact portion and the closure portion are attachable together by attachment means or by a friction fit.

11. An enclosure device according to claim 10, wherein the attachment means comprise one or more of clips, pins, screws, lugs, tabs or latches.

12. An enclosure device according to claim 1, wherein the enclosure device is provided with a flange portion for mounting on the bone surface.

13. An enclosure device according to claim 12, wherein the flange portion is arranged in use to extend from the contact potion or from the closure portion, and is shaped so as to rest on the bone surface.

14. An enclosure device according to claim 12, wherein the flange portion is provided with one or more mounting features to assist in mounting the enclosure device on the bone.

15. An enclosure device according to claim 1 wherein the enclosure device is provided with one or more support portions for supporting the device in use.

16. An enclosure device according to claim 15, wherein the support portions comprise tabs or wings, which have mounting features for mounting on the bone.

17. An enclosure device according to claim 14, wherein the mounting features comprise holes or brackets for receiving fasteners, for example such as screws.

18. An enclosure device according to claim 1, wherein the enclosure device has one or more internal stiffening members for maintaining rigidity of the device.

19. An enclosure device according to claims 1, wherein the enclosure device is provided with one or more loading apertures to allow material to be loaded into the device.

20. An enclosure device according to claim 1, wherein the enclosure device comprises a biocompatible material such as titanium and/or its alloys, ceramics such as calcium hydroxy apatite or polymers such as poly lactic or poly glycolic acids, or combinations thereof.

21. (canceled)
